(19) United States
(12) Patent Application Publication
(10) Pub. No.: US 2012/0196762 A1
(43) Pub. Date: Aug. 2, 2012

(54) METHOD AND APPARATUS FOR DISCOVERY, DEVELOPMENT AND CLINICAL APPLICATION OF MULTIPLEX ASSAYS BASED ON PATTERNS OF CELLULAR RESPONSE

(76) Inventor: Norman A. Paradis, San Marcos, CA (US)

(21) Appl. No.: 13/360,433

(22) Filed: Jan. 27, 2012

Related U.S. Application Data
(60) Provisional application No. 61/436,911, filed on Jan. 27, 2011.

(51) Int. Cl. C12Q 1/02; C40B 30/04; C12Q 1/70; G01N 33/53; C12Q 1/68

(52) U.S. Cl. 506/9; 435/29; 435/7.92; 435/6.12; 435/5

(57) ABSTRACT

A method for deriving a multiplex cell response assay (MCRA), the method comprising:
obtaining at least one specimen that has been phenotyped and classified with respect to the disease of interest using existing diagnostic techniques;
adding of at least one stimulatory or inhibitory agent;
isolating or separating at least one cell type;
performing a multiplex measurement of cellular responses in each of the at least one cell type; and
computationally deriving a clinically useful biomarker algorithm.

### REFERENCE TO PENDING PRIOR PATENT APPLICATION

This patent application claims benefit of pending prior U.S. Provisional Patent Application Ser. No. 61/436,911, filed Jan. 27, 2011 by Norman A. Paradis for MULTIPLEX METHOD FOR DISCOVERY AND CLINICAL APPLICATION OF CELL FUNCTION-BASED BIOMARKER PATTERNS (Attorney's Docket No. BARASH-1 PROV), which patent application is hereby incorporated herein by reference.

### FIELD OF THE INVENTION

This invention relates to assays in general, and more particularly to methods and apparatus for the discovery, development and clinical application of multiplex assays based on patterns of cellular response.

### BACKGROUND OF THE INVENTION

Biomarkers that provide clinically useful results when measured in a univariate manner are uncommon. For this reason, much current genomic, proteomic and gene expression research is directed toward discovery of multivariate patterns that are identified computationally and synthetically multiplexed.

To date, the vast majority of clinically used in-vitro diagnostic assays are proteomic, and are based on measurement of molecular concentration. Investigators have tried to improve and broaden the utility of molecular concentration data through computationally multiplexing the measured concentrations of multiple analytes. Gene and gene expression data have been subjected to similar techniques, with only limited clinical success.

Widely used cellular assays remain uniplex and rudimentary. Most simply count the number of cells present, or use traditional methods to characterize cell type, as with the classical complete blood count. The only significant innovation has been the characterization of cell types based on cell surface patterns of receptors. Most significantly, the vast majority of cellular diagnostics utilize only the number of each cell type and not its function or responsiveness. Even those that measure cellular function or response do so only in a uniplex manner.

An example of such characterization is CD4 (cluster of differentiation 4), a glycoprotein expressed on the surface of T helper cells, monocytes, macrophages, and dendritic cells. Patients with HIV are managed using the CD4 cell counts, but not the functional status of the CD4 cells themselves.

Although there are diagnostic assays that utilize cellular response, they do not synthesize the functional responses of multiple cell types. The Elispot assay, for instance, attempts to characterize clinical status by visual measurement of the cellular production of a single molecular species. It has not been standardized or FDA cleared, and it has not been multiplexed. Flow cytometry simply counts or sorts the types of cells present by cell surface receptors, and suffers from similar limitations.

The crude state of cell-based assays may explain their relatively limited use clinically. More sophisticated assays of greater diagnostic performance accuracy, especially those related to immune cell function, would have significant potential in oncology, rheumatology, infectious disease, and transplantation, among others. Of particular potential might be so-called “companion diagnostic” assays for monoclonal antibodies directed at lymphocytes.

### SUMMARY OF THE INVENTION

In accordance with the present invention, there is provided a method and apparatus for the discovery, development and clinical application of multiplex synthetic biomarker assays based on patterns of the cellular response. After stimulation and or inhibition, selected cell types are assayed for cellular or molecular responses, lack of responses, or changes in state. These are combined into an optimized clinical biomarker using known mathematical or machine learning techniques.

Assays based on the functional response of cells, in particular immune cells, may be multiplexed in multiple dimensions. These may include, but are not limited to, (1) one or more stimulants or inhibitors, (2) one or more cell populations, and (3) one or more cellular responses. Such biomarkers may sometimes hereinafter be referred to as multiplex cell response assays (MCRA).

Because cellular responses are central to homeostasis and disease in metazoans, this technology has broad applications, including but not limited to, (1) as an engine for discovery of multiplex clinical assays based on cellular responses, (2) as multiplex in-vitro clinical assays, (3) research instruments for elucidation of biologic function, and (4) as companion diagnostics for pharmaceuticals.

In one preferred form of the present invention, there is provided a method for deriving a multiplex cell response assay (MCRA), the method comprising:
obtaining at least one specimen that has been phenotyped and classified with respect to the disease of interest using existing diagnostic techniques;
adding of at least one stimulatory or inhibitory agent;
isolating or separating at least one cell type;
performing a multiplex measurement of cellular responses in each of the at least one cell type; and
computationally deriving a clinically useful biomarker algorithm.

### DETAILED DESCRIPTION OF THE PREFERRED EMBODIMENTS

In accordance with the present invention, there is provided a method and apparatus for the discovery, development and clinical application of multiplex synthetic biomarker assays based on patterns of the cellular response. After stimulation and or inhibition, selected cell types are assayed for cellular or molecular responses, lack of responses, or changes in state. These are combined into an optimized clinical biomarker using known mathematical or machine learning techniques.

### Method for Developing The Multiplex Cell Response Assay (MCRA)

In one preferred form of the present invention, the discrete steps in the multiplex cell response assay (MCRA) methods may include:
1. Obtaining whole blood specimens that have been phenotyped for the disease of interest. Such phenotyping may be done on the basis of clinical diagnostics, patient outcomes or the use of established “gold standard” tests.
2. Stimulation of specific cells in whole blood or subfractions of blood. The list of stimulants and inhibitors may include: mitogens, cytokines, chemokines, peptides, glycoproteins, bacterial products, growth factors, phorbyl esters, and cells, among others. More than one stimulant may be used, or a combination of stimulants and inhibitors may be used.
3. Isolation or separation of cell types—the list of cell populations may include any type of cell for which a separation mechanism is available. In particular, these separations are preferably accomplished through binding to cell surface receptors and proteins. Isolation of cell types may be accomplished using an automated cell separator, e.g., the Miltenyi Biotec AutoMACS Separator or the Stemcell Technologies RoboSep machine. Cell populations may be purified based on positive or negative selection strategies using antibodies bound to magnetic microbeads, or other separation methods may be used. An additional refinement of the method may allow for measurement of cellular responses without separation.
4. Multiplex measurement of cellular responses in each of the cell types. Examples of measurable cellular responses may include, but are not limited to, specific metabolites, proteins, or nucleic acids, and alterations in gene expression, the metabolome, proteome, or secreted products, among others.
5. Under some conditions, the steps of stimulation and separation may be may be reversed, such that cell separation precedes cell stimulation. Purified cell populations may then be stimulated with appropriate agents, followed by detection of measurable cellular responses.
6. Computationally deriving cellular response based on diagnostic algorithms built of component cellular responses through training and validation phenotyped samples. The result of phenotyping is utilized by machine learning systems as the “classifying variable” during the training phase of algorithm development.
7. As an engine for discovery, the invention may be employed as a method encompassing a series of separable laboratory steps. Specimen flow from one step to the next need not be automated, it may be manual. Alternatively, future development may allow the whole process, or components, to be accomplished in one reaction chamber.
8. As a multiplex clinical assay(s), the system may be automated to a variable degree. At one end of this range, it may be left as in a form similar to the discovery engine, with each discreet step run by technicians. Progressive automation may include interfacing the components with systems such as the Tecan robotics sample processor all the way through “lab on a chip” technology. The regulatory path may be RUO through IVD.

Multiplex cell response assay (MCRA) systems may comprise four major components, which are optimized according to the specific assay under development:
1. Cell Mixture
2. Stimulant-Suppressant
3. Multiplex Response
4. Mathematical Conversion to an actionable clinical biomarker

It is understood that the order of these steps may be changed or combined.

As a discovery engine, multiplex cell response assay (MCRA) systems may have an additional specimen phenotyping step. It should also be noted that in the discovery and development of individual assays, the constituents of each major component may be optimized using high-throughput techniques, appropriate clinical classifiers and machine learning algorithms.

(1) Cell Mixture

A mixture of immune cells (i.e., T-Cell, B-Cell, Macrophage, among others) and various trophic factors, optimized for the particular assay, may be provided. Various technologies may be used to adjust and optimize the mixture, including, but not limited to, cell sorters, flow cytometry, magnetic beads with monoclonal antibodies, and others.

Cells included in the Cell Mixture may include:
1. Peripheral blood mononuclear cells (PBMC)
2. T-Cell in particular T-Helper, Cytotoxic (CD-8) and others
3. B-Cell
4. Neutrophils
5. Macrophage
6. Dendritic Cell
7. Stem Cell
8. Natural Killer Cell
9. Antigen Presenting Cell

The source of the above cells may be varied, including intravascular, mucosal, and CSF (cerebrospinal fluid) among others. Isolates from tumors or pathologically-affected organs may also be used.

2) Stimulant-Suppressant

A mixture of stimulants and suppressants, also optimized for a particular assay, is then added or co-cultured.

Examples may include:
1. Antigens
2. Mitogens
3. Lymphokines
4. Molecules from bacterial, viral, or fungal sources
5. Endogenous autoimmune related molecules
6. Polyclonal stimuli
7. Growth factors
8. Colony stimulating factors
9. Synthetic peptides or other macromolecules
10. Inhibiting monoclonal antibodies
11. Fluorophores
12. Phorbolmyristate acetate (PMA)
13. Ionomycin
14. Monensin
15. Brefeldin A
16. Sodium chromate for measurement of chromium release
17. Diethylenetriaminepentaacetic acid
18. Adjuvants, such as ISCOMS and Incomplete Freund's Adjuvant (IFA)

The concentrations of any of the above stimulants/suppressants may be varied. The constituents of the cell culture media may be varied. There can also be variation in the incubation period or the physical conditions of incubation.

Initial components that may be evaluated experimentally for inclusion in either the Cell Mixture or Stimulant-Suppressant Mixture may be chosen empirically, based on a current understanding of the pathophysiology of the disease in question. A large number of components may be evaluated experimentally for inclusion in the Multiplex Response pattern because of the availability of high-throughput techniques.

3) Multiplex Response

The multiplex cellular response pattern is then measured. This pattern of response may be made up of measurement of:
1. Antibodies
2. Lymphokines
3. Chemokines
4. Intracellular protein staining
5. Interleukins such as IL-2, IL-4, IL-6, IL-12
6. Interferons such as IFN-γ
7. Cell surface markers: CD 25, CD 69, CD 71, HLA-DR
8. Components of cell signaling cascades
9. Patterns of gene expression
10. RNA by PCR
11. Enzyme-linked immunospot (ELISPOT)
12. Patterns of RNA inhibition
13. Fluorometry
14. Cell signaling pathways
15. Measurement of compliment
16. Indicators of B or T cell activation
17. Indicators of Stem cell activation
18. Indicators of NK cell activation
19. Indicators of cell-tissue mobilization—integrins
20. Indicators of apoptosis or necrosis
21. Indicators of hematopoiesis
22. fluorescence staining
23. Quantification using limiting dilution assays
24. Colorimetric measurement
25. TNF-α
26. Indicators of MHC-peptide binding such as the tetramer assay
27. Any of the above compared to a control
28. Indicator substances for any of the above
29. Kinetic patterns for any of the above
30. Concentration patterns for any of the above
31. Concentration and kinetic patterns combined for any of the above

The response patterns can be measured in a number of different manners:
1. Lymphopoliferation—measured by any of a number of mechanisms including radiolabelled thymidine
2. ELISA and similar assays
3. High-throughput genomics and proteomics
4. Mass spectroscopy

4) Mathematical Conversion to an actionable clinical biomarker (an assay)

Techniques for development of multiplex algorithms are well known (see, for example, Kato K. Algorithm for in vitro diagnostic multivariate index assay. Breast Cancer 2009; 16(4):248-251), and include multivariate and multivariable analysis, machine learning, and data mining techniques, multivariable regression, neural networks, and nearest neighbor algorithms, among others. Any of the techniques used in development of in vitro diagnostic multivariate index assays would be applicable to the development of MCRAs. In particular, supervised statistical techniques based on existing clinical classifiers will utilized.

During the discovery phase, it is anticipated that more than one of these techniques will be evaluated for its ability to derive the best and most efficient clinical biomarker. In particular, cross validation techniques such as “leave-one-out” may be used to iteratively build the new synthetic classifier. The model or algorithm that produces the optimal diagnostic performance is selected. In particular, the objective is to identify the most accurate diagnostic algorithm based on the fewest number of input variables. Once the multiplex algorithm is developed, it is preferably converted to an index for ease of clinical use.

### Implementation of The Method For Developing the Multiplex Cell Response Assay (MCRA)

The foregoing method for developing the multiplex cell response assay (MCRA) may be implemented using various dedicated and/or programmable machines, e.g., the foregoing method for developing the MCRA may be implemented using a general purpose computer appropriately programmed to derive the MCRA.

### Examples of Multiplex Cell Response Assay

In patients under evaluation for possible acute or chronic infection, MCRAs may be used to better delineate the relationship between the infectious organism and the patient's possible immune response. Is there active infection? Is there previous exposure with or without latent disease? Is there going to be an effective clinical response? In this situation, the Cell Mixture may include cell types known to be functional in the immune response to the disease in question. The Stimulant-Suppressant Mixture may include a combination of bacterial, viral or fungal antigens/epitopes specific to the disease or potential diseases in question. The measured Multiplex Response may include cytokines, lymphokines or interferons related to infection with, or immunity to, the disease in question.

Assay for TB exposure, immunity or current active disease. In this situation, the Cell mixture may include cell types known to be functional in the immune response to the tubercle bacillus such as cytotoxic cells. The Stimulant-Suppressant Mixture may include a combination of tubercle bacillus antigens or even whole bacillus such as BCG.

The measured multiplex response may include cytokines in dictated of immunity or infection such as gamma interferon or IL-12.

Viral Infection, Reactivation or Immune Status. In this situation, the Cell Mixture may include cell types known to be functional in the immune response to the virus in question such as T-Helper and Cytotoxic-T cells. The Stimulant-Suppressant Mixture may include a combination of viral subtypes, epitopes, or even whole virus. The measured multiplex response may include cytokines indicative of immunity, reactivation or infection such as gamma interferon or IL-12, or peptide-loaded MHC complexes. In addition to the examples described above, the MCRA may be developed for any viral illness.

Fungal Infection, Reactivation or Immune Status. Delineating the clinical status of patients potentially infected with fungal species such as Candida, histoplasmosis, aspergillosis, and others is particularly challenging for clinicians. These may be dormant or active depending on the immune status of the patient. In this situation, the Cell Mixture may include cell types known to be functional in the immune response to fungal pathogens such as macrophages, T-Cells, and neutrophils. The Stimulant-Suppressant Mixture may include a combination of fungal epitopes, or even whole fungus. Also included may be ligands for receptors that initiate innate immunity. The measured Multiplex Response may include cytokines indicative of immunity, reactivation or infection such as gamma interferon or IL-12.

Cytomegalovirus (CMV), Infection, Reactivation or Immune Status. In this situation, the Cell Mixture may include cell types known to be functional in the immune response to CMV such as T-Helper and Cytotoxic-T cells. The Stimulant-Suppressant Mixture may include a combination of CMV epitopes, CMV pp65 and IE-1 proteins, or even whole virus. The measured Multiplex Response may include cytokines indicative of immunity, reactivation or infection such as gamma interferon or IL-12, or peptide-loaded MHC complexes.

Herpes Simplex Virus (HSV) Infection, Reactivation or Immune Status. In this situation, the Cell Mixture may include cell types known to be functional in the immune response to HSV such as T-Helper and Cytotoxic-T cells. Stimulant-Suppressant Mixture may include a combination of HSV subtypes, epitopes, or even whole virus. The measured Multiplex Response may include cytokines indicative of immunity, reactivation or infection such as gamma interferon or IL-12, or peptide-loaded MHC complexes.

HIV Infection, Reactivation or Immune Status. In this situation, the Cell Mixture may include cell types known to be functional in the immune response to HIV such as T-Helper and Cytotoxic-T cells. The Stimulant-Suppressant Mixture may include a combination of HIV subtypes, epitopes, or even whole virus. The measured Multiplex Response may include cytokines indicative of immunity, reactivation or infection such as gamma interferon or IL-12, or peptide-loaded MHC complexes.

Vaccine Response. CMI is central to the efficacy of vaccines. In developing a MCRA for vaccine response measurement, the Cell Mixture may include cell types known to be functional in the immune response induced by the vaccine, such as T-Helper and Cytotoxic-T cells. Stimulant-Suppressant Mixture may include a combination of epitopes that constitute the vaccine. The measured Multiplex Response may include indicators of immune cell response.

Cancer and Cancer Vaccines Assays. Host CMI is likely important to the outcome in patients with cancer, and is the basis of efficacy of cancer vaccines. In developing a MCRA for vaccine response measurement, the Cell Mixture may include cell types known to be functional in the immune response to cancer in question such as dendritic cells, CD-8, T-Helper, Cytotoxic-T cells or NKC. The Stimulant-Suppressant Mixture may include a combination of antigens derived from oncogenes, overexpressed genes, embryonic genes, normal differentiation genes, viral genes (HPV), tumor-suppressor genes (p53), and other tumor-associated proteins (MUC1). Tumor-derived RNA, apoptotic bodies, and lysates may also be used. The measured Multiplex Response may include cytokines indicative of immunity, reactivation or infection such as gamma interferon or IL-12, or peptide-loaded MHC complexes.

Neurological Diseases such as Multiple Sclerosis, Alzheimer's and Others. Many neurological diseases, such as MS, have CMI as an intrinsic component of their pathophysiology. In developing a MCRA for a neurological disease, the Cell Mixture may include cell types known to be involved in the disease process itself and these may best be obtained from cerebrospinal fluid. The Stimulant-Suppressant Mixture may include a combination of proteins also known to be involved in the disease. In the case of MS, this may be myelin basic protein or a subset of its epitopes. The measured Multiplex Response may include indicators of immune cell response.

Allergy Tests. Delineating the clinical status of patients potentially suffering with allergic illness is also particularly challenging for clinicians. The range of illness includes mucosal inflammation, dermatitis, anaphylaxis, etc., and cannot be confused with illnesses of other etiology. MCRAs might be particularly useful in the evaluation of allergic patients. In this situation, the Cell Mixture may include cell types known to be important in allergy such as Ig-E producing B Cells, but also including macrophages, T-Cells. Stimulant-Suppressant Mixture may include a combination of potentially allergic epitopes. The measured Multiplex Response may include Ig-E, histamine, complement, among others.

These examples are intended to augment the description of some possible MCRA. It is understood that the system is a generalizable platform that will likely allow development of clinical diagnostic assays in almost any area of medicine.

### Modifications Of The Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A method for deriving a multiplex cell response assay (MCRA), the method comprising:
obtaining at least one specimen that has been phenotyped and classified with respect to the disease of interest using existing diagnostic techniques;
adding of at least one stimulatory or inhibitory agent;
isolating or separating at least one cell type;
performing a multiplex measurement of cellular responses in each of the at least one cell type; and
computationally deriving a clinically useful biomarker algorithm.

2. A method according to claim 1 further comprising repeating the foregoing steps iteratively so as to optimize performance of the algorithm.

3. A method according to claim 1 further comprising generating an index reflective of the disease of interest.

4. A method according to claim 1 wherein the at least one cell type is involved in an immune response.

5. A method according to claim 1 wherein the at least one cell type is selected from the group consisting of peripheral blood mononuclear cells (PBMC), T-Helper, Cytotoxic (CD-8) cell, Neutrophil, Dendritic Cell, Stem Cell, Natural Killer Cell, Antigen Presenting Cell.

6. A method according to claim 1 wherein the at least one specimen is whole blood or a subfraction such as serum or plasma.

7. A method according to claim 1 wherein the at least one stimulatory or inhibitory agent is a cytokine or a chemokine

8. A method according to claim 1 wherein the at least one stimulatory or inhibitory agent contains an immunological epitope of interest.

9. A method according to claim 1 wherein a plurality of stimulatory or inhibitory agents are used.

10. A method according to claim 1 wherein a combination of stimulatory and inhibitory are used.

11. A method according to claim 1 wherein the at least one stimulatory or inhibitory agent is selected from the group consisting of: an antigen, a mitogen, a lymphokine, molecules from bacterial, viral, or fungal sources, endogenous autoimmune related molecules, a growth factor, a colony stimulating factor, synthetic peptides or other macromolecules, inhibiting antibodies, phorbolmyristate acetate (PMA), Ionomycin, Monensin, brefeldin A, diethylenetriaminepentaacetic acid, or an adjuvant such as ISCOMS or incomplete Freund's Adjuvant.

12. A method according to claim 1 wherein the order of the component steps is changed.

13. A method according to claim 1 wherein the isolating or separating of cell types is performed before adding of at least one stimulatory or inhibitory agent.

14. A method according to claim 1 wherein the at least one stimulatory or inhibitory agent comprises a combination of bacterial, viral or fungal antigens/epitopes specific to the diseases in question.

15. A method according to claim 1 wherein the measured cellular response is the identification of the presence of a metabolite.

16. A method according to claim 1 wherein the measured cellular response is the identification of the presence of a macromolecule, such as a protein, lipid or nucleic acid.

17. A method according to claim 1 wherein the measured cellular response a the identification of the presence of a combination of molecules.

18. A method according to claim 1 wherein the measured cellular response is the identification of a pattern of gene expression or related to a pattern of gene expression.

19. A method according to claim 1 wherein the measured cellular response is the identification of the concentration of at least one selected from the group consisting of: lymphokines; chemokines; interleukins, interferons, cell surface markers, components of cell signaling cascades; ribonucleic acid, compliment, indicators of B or T cell activation, indicators of stem cell activation, indicators of NK cell activation, indicators of cell-tissue mobilization—integrins, indicators of apoptosis or necrosis, indicators of hematopoiesis, or indicators of MHC-peptide binding.

20. A method according to claim 1 wherein the cellular response is measured using at least one from the group consisting of: intracellular protein staining, RNA by PCR, Enzyme-linked immunospot (ELISPOT), fluorometry, fluorescence staining, quantification using limiting dilution assays, colorimetric measurement, indicator substances, kinetic patterns, concentration patterns, lymphoproliferation, radiolabelling, ELISA and similar assays, high-throughput genomics and proteomics, mass spectroscopy.

21. A method according to claim 3 wherein the index comprises a single diagnostic result indicative of the likelihood of the presence of the disease of interest.

22. A method according to claim 1 wherein a single classifier is used to derive the algorithm.

23. A method according to claim 1 wherein multiple classifiers, each weighted differently, are used to derive the algorithm.

24. A method according to claim 1 wherein at least one of the classifiers is a clinical presentation or outcome.

25. A method according to claim 1 wherein at least one of the classifiers is a laboratory derived measurement.

26. A method according to claim 1 wherein at least one of the cell types is obtained after administration of physical or pharmacologic agents whose physiologic effects on the probability distribution is favorable to diagnostic performance.

27. A method according to claim 1 wherein the assay is utilized to identify patients with acute or chronic infection through stimulation with appropriate epitopes from the organism of interest.

28. A method according to claim 1 wherein the assay is utilized to determine if there has been an effective clinical response in an infected patient after treatment through stimulation with appropriate epitopes from the organism of interest.

29. A method according to claim 1 wherein the assay is utilized to determine if the patient has TB exposure, immunity or current active disease.

30. A method according to claim 1 wherein the assay is utilized to determine if viral infection, reactivation or immunity has occurred.

31. A method according to claim 1 wherein the assay is utilized to determine if fungal infection, reactivation or immunity has occurred.

32. A method according to claim 1 wherein the assay is utilized to determine if Cytomegalovirus (CMV) infection, reactivation or immunity has occurred.

33. A method according to claim 1 wherein the assay is utilized to determine if herpes simplex virus (HSV) infection, reactivation or immunity has occurred.

34. A method according to claim 1 wherein the assay is utilized to determine if human immunodeficiency virus infection, reactivation or immunity has occurred.

35. A method according to claim 1 wherein the assay is utilized to determine if an effective immune response has occurred after vaccination.

36. A method according to claim 1 wherein the assay is utilized to determine if an effective immune response has occurred after immunization for the treatment of cancer.

37. A method according to claim 1 wherein the assay is utilized to determine if a cancer has developed, progressed, regressed, gone into remission or been cured.

38. A method according to claim 1 wherein the assay is utilized to determine if a patient is at risk or is developing a neurological disease such as multiple sclerosis, Alzheimer's, Parkinson's, or others, and the effect of treatment.

39. A method according to claim 1 wherein the assay is utilized to determine if a patient is allergic to a specific allergen, the degree of allergy, or the response to treatment of an allergy.
